(19) United States
(12) Patent Application Publication
(10) Pub. No.: US 2005/0256097 A1
(43) Pub. Date: Nov. 17, 2005

(54) Pharmaceutical solution formulations containing 17-AAG

(75) Inventors: Ziyang Zhong, Union City, CA (US); Peter J. Licari, Fremont, CA (US)

Correspondence Address: KOSAN BIOSCIENCES, INC, 3832 BAY CENTER PLACE, HAYWARD, CA, 94588, US

(73) Assignee: Kosan Biosciences, Inc., Hayward, CA (US)

(21) Appl. No.: 11/123,570

(22) Filed: May 5, 2005

Related U.S. Application Data
(60) Provisional application No. 60/570,215, filed on May 11, 2004.

(51) Int. Cl. A61K 31/33; A61K 31/20

(52) U.S. Cl. 514/183; 514/560

(57) ABSTRACT

A pharmaceutical solution formulation containing 17-AAG in an amount of up to 15 mg/mL dissolved in a vehicle comprising (i) a first component that is ethanol, in an amount of between about 40 and about 60 volume %; (ii) a second component that is a polyethoxylated castor oil, in an amount of between about 15 to about 50 volume %; and (iii) a third component that is selected from the group consisting of propylene glycol, PEG 300, PEG 400, glycerol, and combinations thereof, in an amount of between about 0 and about 35 volume %.

### CROSS-REFERENCE TO RELATED APPLICATIONS

This application claims the benefit under 35 U.S.C. §119(e) of U.S. Provisional Application No. 60/570,215, filed May 11, 2004, the disclosure of which is incorporated herein by reference.

### BACKGROUND OF THE INVENTION

1. Field of the Invention

This invention relates to pharmaceutical solution formulations containing 17-allylamino-17-demethoxygeldanamycin (“17-AAG”) and methods for their preparation and use.

2. Description of Related Art

Geldanamycin belongs to the ansamycin family of natural products, whose members are characterized by a benzenoid nucleus (typically a benzoquinone or hydroquinone nucleus) connected at two meta positions to form a macrolactam. Besides geldanamycin, the ansamycins include the macbecins, the herbimycins, the TAN-420s, and reblastatin.

Geldanamycin and its derivatives are the most extensively studied of the ansamycins. Although geldanamycin was originally was identified as a result of screening for antibiotic activity, current interest in it is based primarily on its cytotoxicity towards tumor cells and, therefore, its potential as an anticancer agent. It is an inhibitor of heat shock protein-90 (“Hsp90”), which is involved in the folding, activation and assembly of a wide range of proteins (“client proteins”), including key proteins involved in signal transduction, cell cycle control and transcriptional regulation. The binding of geldanamycin to Hsp90 disrupts Hsp90-client protein interactions, preventing the client proteins from folding correctly and rendering them susceptible to proteasome-mediated destruction. Among the Hsp90 client proteins are many mutated or overexpressed proteins implicated in cancer: p53, Bcr-Abl kinase, Raf-1 kinase, Akt kinase, Npm-Alk kinase p185^{ErB2} transmembrane kinase, Cdk4, Cdk6, Wee1 (a cell cycle-dependent kinase), HER2/Neu (ErbB2), and hypoxia inducible factor-1α (HIF-1α). However, the hepatotoxicity and poor bioavailability of geldanamycin have lead to its discontinuation as a clinical candidate.

Nevertheless, interest persists in the development of geldanamycin derivatives or analogs having geldanamycin-like bioactivity, but with a more pharmaceutically acceptable spectrum of properties. Position 17 of geldanamycin has been an attractive focal point, chemically speaking, for the synthesis of geldanamycin derivatives because the methoxy group there is readily displaced by a nucleophile, providing a convenient entry into 17-substituted-17-demethoxygeldanamycins. Further, structure-activity relationship (SAR) studies have shown that chemically and sterically diverse 17-substituents can be introduced without destroying antitumor activity. See, e.g., Sasaki et al., U.S. Pat. No. 4,261,989 (1981); Schnur et al., U.S. Pat. No. 5,932,566 (1999); Schnur et al., J. Med. Chem., 38, 3806-3812 (1995); Schnur et al., J. Med. Chem., 38, 3813-3820 (1995); and Santi et al., U.S. 2003/0114450 A1 (2003); the disclosures of which are incorporated by reference. The SAR inferences are supported by the X-ray crystal co-structure of the complex between Hsp90 and a geldanamycin derivative (17-DMAG, v. infra), showing that the 17-substituent projects out from the binding pocket and into the solvent (Jez et al., Chemistry & Biology, 10, 361-368 (2003)). Thus, position 17 is an attractive one for the introduction of property-modulating substituents, such as a solubilizing group. The best-known 17-substituted geldanamycin derivative is 17-AAG, first disclosed in Sasaki et al., cited supra, and currently undergoing clinical trials. Another noteworthy 17-substituted geldanamycin derivative is 17-(2-dimethylaminoethyl)amino-17-demethoxygeldanamycin (“17-DMAG”, Snader et al., 2004/0053909 A1 (2004)), also undergoing clinical trials.

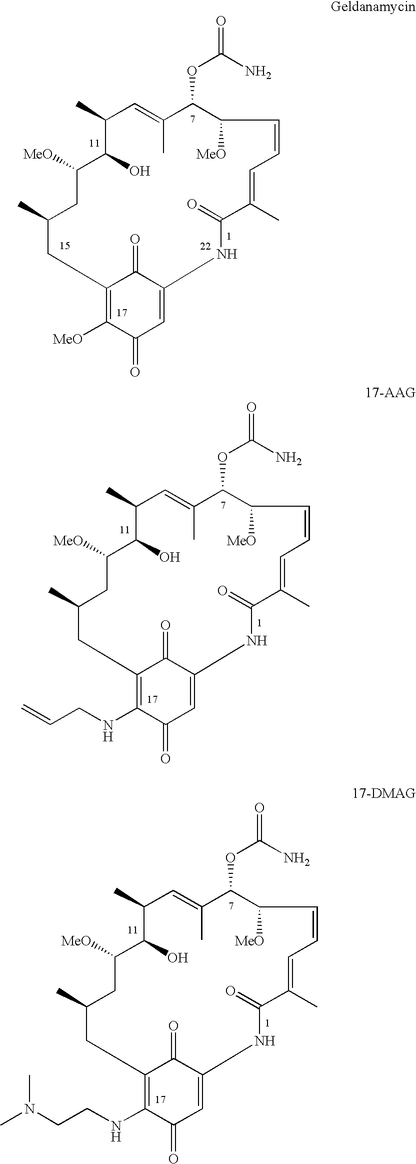

A limitation in the preparation of pharmaceutical formulations containing geldanamycin compounds such as geldanamycin itself and 17-AAG, especially for parenteral administration, is their very poor water solubility, only about 0.1 mg/mL at neutral pH for 17-AAG. (17-DMAG, having an alkyl amino group, is more soluble.) Addressing this issue, Tabibi et al., U.S. Pat. No. 6,682,758 B1 (2004) disclosed a formulation for a water insoluble drug such as 17-AAG comprising (a) the drug, (b) a water-miscible organic solvent for the drug, (c) a surfactant, and (d) water. The water miscible solvent can be dimethylsulfoxide (DMSO), dimethylformamide, ethanol, glycerin, propylene glycol, or polyethylene glycol. The surfactant preferably is a phospholipid (especially egg phospholipid). Another disclosure of interest is Ulm et al., WO 03/086381 (2003), which discloses a method for preparing pharmaceutical formulations for ansamycins by (a) providing the ansamycin dissolved in ethanol; (b) mixing the product of step (a) with a medium chain triglyceride to form a first mixture; (c) substantially removing the ethanol from the first mixture; (d) combining the product of step (c) with an emulsifying agent and a stabilizer to form a second mixture; and (e) emulsifying the second mixture. The emulsified second mixture optionally can be lyophilized and then re-hydrated. In a specific combination, the medium chain triglyceride comprises caprylic and/or caproic acid, the emulsifying agent comprises phosphotidylcholine, and stabilizer comprises sucrose. Additionally, Ulm et al., WO 2004/082676 A1 (2004) discloses a pharmaceutical composition comprising an Hsp90 inhibitor such as 17-AAG, an emulsifying agent, and an oil comprising both medium and long chain triglycerides.

### BRIEF SUMMARY OF THE INVENTION

In one aspect, the present invention provides an improved solution formulation for 17-AAG, suitable for intravenous administration. Such formulation comprises 17-AAG in an concentration of up to 15 mg/mL dissolved in a vehicle comprising (i) a first component that is ethanol, in an amount of between about 40 and about 60 volume %; (ii) a second component that is a polyethoxylated castor oil, in an amount of between about 15 to about 50 volume %; and (iii) a third component that is selected from the group consisting of propylene glycol, PEG 300, PEG 400, glycerol, and combinations thereof, in an amount of between about 0 and about 35 volume %. The aforesaid percentages are volume/volume percentages based on the combined volumes of the first, second, and third components. The lower limit of about 0 volume % for the third component means that it is an optional component; that is, it may be absent.

In another aspect, this invention provides a method for administering 17-AAG to a patient in need thereof, comprising the steps of:
(a) providing a pharmaceutical solution formulation comprising 17-AAG in concentration of up to 15 mg/mL dissolved in a vehicle comprising (i) a first component that is ethanol, in an amount of between about 40 and about 60 volume %; (ii) a second component that is a polyethoxylated castor oil, in an amount of between about 15 to about 50 volume %; and (iii) a third component that is selected from the group consisting of propylene glycol, PEG 300, PEG 400, glycerol, and combinations thereof, in an amount of between about 0 and about 35 volume %;
(b) diluting the pharmaceutical solution formulation of step (a) into water to prepare a diluted formulation containing up to 3 mg/mL 17-AAG; and
(c) administering the diluted formulation intravenously to a patient.

In yet another embodiment, there is provided a method for preparing a pharmaceutical solution formulation comprising 17-AAG, comprising the steps of:
(a) providing an amount of 17-AAG;
(b) combining the 17-AAG of step (a) with an amount of a vehicle comprising (i) a first component that is ethanol, in an amount of between about 40 and about 60 volume %; (ii) a second component that is a polyethoxylated castor oil, in an amount of between about 15 to about 50 volume %; and (iii) a third component that is selected from the group consisting of propylene glycol, PEG 300, PEG 400, glycerol, and combinations thereof, in an amount of between about 0 and about 35 volume %;
(c) stirring the combination from step (b) until the 17-AAG is substantially dissolved; and
(d) optionally filtering the stirred combination from step (c) to form a pharmaceutical solution formulation comprising 17-AAG;
   the amount of 17-AAG in step (a) and the amount of vehicle in step (b) being such that the concentration of 17-AAG in the pharmaceutical solution formulation is up to 15 mg/mL.

### DETAILED DESCRIPTION OF THE INVENTION

The pharmaceutical solution formulation of this invention is stable, forming a clear purple solution, and can be conveniently diluted into water for injection (“WFI”) to form a clear diluted formulation containing up to 3 mg/mL 17-AAG (preferably between 0.2 and 3 mg/mL), suitable for intravenous injection. The diluted formulation is stable for a period of time, at least 10 hrs and usually approximately 12 to 24 hours. Prolonged storage of the diluted formulation is not recommended, due to stability and sterility issues. Administration of undiluted formulation is not recommended.

Compared to prior art formulations, the present pharmaceutical solution formulation offers a number of advantages. It is easily prepared and stored and does not require multiple solvent addition, removal and/or re-addition steps, other than the final dilution into WFI prior to use. It avoids the use of a solvent such as DMSO, which has poor patient acceptance because of its odor (or that of its metabolite(s)). The present pharmaceutical solution formulation allows delivery of the requisite amount of 17-AAG within an acceptable infusion time, ca. 90 min.

Preferably, the vehicle comprises ethanol (first component) in an amount of about 50 volume %, polyethoxylated castor oil (second component) in an amount of between about 20 to about 30 volume %, and propylene glycol as the third component, in an amount of between about 20 and about 30 volume %.

The propylene glycol can be replaced entirely or in part by PEG 300 (300 average molecular weight poly(ethylene glycol)), PEG 400 (400 average molecular weight poly(ethylene glycol)), glycerol, or combinations thereof.

The ethanol is preferably dehydrated USP grade. The propylene glycol, PEG 300, PEG 400, or glycerol is preferably USP grade.

The polyethoxylated castor oil acts as a solubilizer/emulsifier for the 17-AAG. Preferably, the polyethoxylated castor oil is that produced by BASF AG under the trade name Cremophor. Particularly preferred is Cremophor EL, although other grades of Cremophor, such as Cremophor RH 60, Cremophor CO 40, Cremophor CO 410, Cremophor CO 455, Cremophor CO 60, Cremophor RH 40, Cremophor RH 410 and Cremophor WO 7 may be used. Those skilled in the art will appreciate that Cremophor-based formulations should be used with a certain degree of care, as some patients have experienced adverse side effects.

Although various grades of Cremophor have been used as formulation aids in respect of pharmaceuticals, Cremophor has not hitherto used with ansamycins. In fact, the use of Cremophor in ansamycin formulations was recommended against in Santi et al., U.S. 2003/0114450 A1 (2003). By way of background, illustrative disclosures of Cremophor-containing formulations involving other pharmaceuticals include: Brahm, U.S. Pat. No. 5,583,153 (1996); Gao et al., U.S. Pat. No. 6,121,313 (2000); Kuo et al., U.S. Pat. No. 6,214,803 B1 (2001); Chen et al., U.S. Pat. No. 6,555,558 B2 (2003); Xiang et al., U.S. Pat. No. 6,653,319 B1 (2003); Whittle et al., U.S. 2003/0021752 A1 (2003); Gao et al., U.S. 2003/0044434 A1 (2003); Jiang et al., U.S. 2003/0091639 A1 (2003); Hauer et al., U.S. 2003/0104990 A1 (2003); Cai et al., U.S. 2003/0114485 A1 (2003); Stanislaus, U.S. 2003/0119909 A1 (2003); Naicker et al., U.S. 2003/0171264 A1 (2003); Dong et al., U.S. 2003/0198619 A1 (2003); Dong et al., U.S. 2003/0232078 A1 (2003); Metcalfe et al., U.S. 2004/0033243 A1 (2004); Namburi et al., U.S. 2004/0052847 A1 (2004); and Danishefsky et al., U.S. 2004/0053910 A1 (2004). The disclosures of the foregoing documents are incorporated herein by reference.

In the preparation of the vehicle, the first, second, and third components preferably are combined in the order recited, as detailed hereinbelow. That is, the first component is combined with the second component, after which the third component is added to the combined first and second components.

After the vehicle has been prepared, the pharmaceutical solution formulation can be prepared as follows: A pre-measured amount of 17-AAG is weighed into an appropriate container, to which is then added a pre-measured amount of vehicle. The 17-AAG and vehicle are then stirred until the 17-AAG is dissolved (preferably for at least 6 hr, more preferably for at least 10 hr, most preferably for 12 to 14 hr or overnight) and filtered, preferably through a 0.22μ filter, to provide a pharmaceutical solution formulation of this invention. The stirring may be at ambient temperature or under refrigeration. Once made, the formulation preferably is stored under refrigeration, preferably at a temperature between −20 and 4° C. Use of brown glass vials or other suitable containers to protect the 17-AAG from light is recommended. As mentioned above, the concentration of 17-AAG can be up to 15 mg/mL and preferably is between 2 and 15 mg/mL.

The vehicle is said to comprise the first, second, and third components, meaning that it is amenable to the inclusion of further ingredients. However, in a preferred embodiment the vehicle consists essentially of the first, second and third components in the aforementioned relative amounts, by which is meant that the vehicle is limited to the afore-specified three components and those that do not materially affect the basic and novel characteristic(s) of the pharmaceutical solution formulation of this invention.

Geldanamycin is a well-known natural product, obtainable by culturing the producing organism, Streptomyces hygroscopicus var. geldanus NRRL 3602. 17-AAG is made semi-synthetically from geldanamycin, by reaction of geldanamycin with allylamine, as described in Sasaki et al., U.S. Pat. No. 4,261,989 (1981), the disclosure of which is incorporated herein by reference.

17-AAG administered via a pharmaceutical solution formulation of this invention can be used for treating diseases such as, but not limited to, hyperproliferative diseases, including: cancers of the head and neck which include tumors of the head, neck, nasal cavity, paranasal sinuses, nasopharynx, oral cavity, oropharynx, larynx, hypopharynx, salivary glands, and paragangliomas; cancers of the liver and biliary tree, particularly hepatocellular carcinoma; intestinal cancers, particularly colorectal cancer; treat ovarian cancer; small cell and non-small cell lung cancer; breast cancer sarcomas, such as fibrosarcoma, malignant fibrous histiocytoma, embryonal rhabdomysocarcoma, leiomysosarcoma, neurofibrosarcoma, osteosarcoma, synovial sarcoma, liposarcoma, and alveolar soft part sarcoma; neoplasms of the central nervous systems, particularly brain cancer; lymphomas such as Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma. Clinically, practice of the methods and use of compositions described herein will result in a reduction in the size or number of the cancerous growth and/or a reduction in associated symptoms (where applicable). Pathologically, practice of the method and use of compositions described herein will produce a pathologically relevant response, such as: inhibition of cancer cell proliferation, reduction in the size of the cancer or tumor, prevention of further metastasis, and inhibition of tumor angiogenesis. The method of treating such diseases comprises administering a therapeutically effective amount of an inventive combination to a subject. The method may be repeated as necessary.

Non-cancer disorders that are characterized by cellular hyperproliferation can also be treated by 17-AAG administered in accordance with this invention. Illustrative examples of such disorders include but are not limited to: atrophic gastritis, inflammatory hemolytic anemia, graft rejection, inflammatory neutropenia, bullous pemphigoid, coeliac disease, demyelinating neuropathies, dermatomyositis, inflammatory bowel disease (ulcerative colitis and Crohn's disease), multiple sclerosis, myocarditis, myositis, nasal polyps, chronic sinusitis, pemphigus vulgaris, primary glomerulonephritis, psoriasis, surgical adhesions, stenosis or restenosis, scleritis, scleroderma, eczema (including atopic dermatitis. irritant dermatitis, allergic dermatitis), periodontal disease (i.e., periodontitis), polycystic kidney disease, and type I diabetes. Other examples include vasculitis (e.g., Giant cell arteritis (temporal arteritis, Takayasu's arteritis), polyarteritis nodosa, allergic angiitis and granulomatosis (Churg-Strauss disease), polyangitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system, Wegener's granulomatosis, Kawasaki's disease, vasculitis of the central nervous system, Buerger's disease and systemic sclerosis); gastrointestinal tract diseases (e.g., pancreatitis, Crohn's disease, ulcerative colitis, ulcerative proctitis, primary sclerosing cholangitis, benign strictures of any cause including ideopathic (e.g., strictures of bile ducts, esophagus, duodenum, small bowel or colon); respiratory tract diseases (e.g., asthma, hypersensitivity pneumonitis, asbestosis, silicosis and other forms of pneumoconiosis, chronic bronchitis and chronic obstructive airway disease); nasolacrimal duct diseases (e.g., strictures of all causes including ideopathic); and eustachean tube diseases (e.g., strictures of all causes including ideopathic).

17-AAG can be administered in combination with other anti-cancer or cytotoxic agents, including alkylating agents, angiogenesis inhibitors, antimetabolites, DNA cleavers, DNA crosslinkers, DNA intercalators, DNA minor groove binders, heat shock protein 90 inhibitors, histone deacetylase inhibitors, microtubule stabilizers, nucleoside (purine or pyrimidine) analogs, proteasome inhibitors, topoisomerase (I or II) inhibitors, tyrosine kinase inhibitors. Specific anti-cancer or cytotoxic agents include β-lapachone, 17-DMAG, bicalutamide, bleomycin, bleomycin, bortezomib, busulfan, calicheamycin, camptothecin, capecitabine, callistatin A, CC-1065, cisplatin, cryptophycins, daunorubicin, discodermolide, docetaxel, doxorubicin, duocarmycin, dynemycin A, epothilones, etoposide, floxuridine, floxuridine, fludarabine, fluoruracil, gefitinib, geldanamycin, gemcitabine, hydroxyurea, imatinib, interferons, interleukins, irinotecan, leptomycin B, methotrexate, mitomycin C, oxaliplatin, paclitaxel, spongistatins, suberoylanilide hydroxamic acid (SAHA), thiotepa, topotecan, trichostatin A, vinblastine, vincristine, and vindesine.

The co-administered anti-cancer or cytotoxic agent can be a protein kinase inhibitor, including: quinazolines, particularly 4-anilinoquinazolines such as Iressa (AstraZeneca; N-(3-chloro-4-fluorophenyl)-7-methoxy-6-[3-(4-morpholinyl)propoxy]-4-quinazolinamine) and Tarceva (Roche/Genentech; N-(3-ethynylphenyl)-6,7-bis(2-methoxyethoxy)-4-quinazolinamine monohydrochloride); phenylamino-pyrimidines such as Gleevec (Novartis; 4-[(4-methyl-1-piperazinyl)methyl]-N-[4-methyl-3-[[4-(3-pyridinyl)-2-pyrimidinyl]amino]phenyl]benzamide); pyrrolo- and pyrazolopyrimidines such as BIBX 1382 (Boehringer Ingelheim; N8-(3-chloro-4-fluorophenyl)-N-2-(1-methyl-4-piperidinyl)-pyrimido[5,4-d]pyrimidine-2,8-diamine); indoles and oxindoles such as Semaxinib (Pharmacia; 3-[(3,5-dimethyl-1H-pyrrol-2-yl)methylene]-1,3-dihydro-2H-Indol-2-one); benzylidene malononitriles; flavones such as flavopiridol (Aventis; 2-(2-chlorophenyl)-5,7-dihydroxy-8-[(3S,4R)-3-hydroxy-1-methyl-4-piperidinyl]-4H-1-benzopyran-4-one); staurosporines such as CEP-701 (Cephalon); antibodies such as Herceptin (Genentech); and ribozymes such as Angiozyme (Ribozyme Pharmaceuticals).

Using a pharmaceutical solution formulation of this invention, 17-AAG may be administered in a dose ranging from about 4 mg/m² to about 4000 mg/m², depending on the frequency of administration. A preferred dosage regimen for 17-AAG is about 450 mg/m² weekly (Banerji et al., Proc. Am. Soc. Clin. Oncol., 22, 199 (2003, abstract 797), “A Pharmacokinetically (PK)-pharmacodynamically (PD) Guided Phase I Trial of the Heat Shock Protein 90 (HSP90) Inhibitor 17-Allyl-17-demethoxygeldanamycin (17AAG)”). Alternatively, a dose of about 308 mg/m² weekly can be administered. See Goetz et al., Eur. J. Cancer, 38 (Supp. 7), S54-S55 (2002), “A phase I trial of 17-Allyl-Amino-Geldanamycin (17-AAG) in patients with advanced cancer.” Another dosage regimen is twice weekly, with doses ranging from 220 mg/m² to 340 mg/m² (preferably either 220 mg/m² or 340 mg/m²). A dosage regimen that can be used for combination treatments with another drug, such as docetaxel, is to administer the two drugs every three weeks, with the dose of 17-AAG being up to 650 mg/m² at each administration.

The practice of this invention can be further understood by reference to the following examples, which are provided by way of illustration and not of limitation.

### EXAMPLE 1

This example describes the preparation of a vehicle for use in formulations of this invention, consisting of 50 volume % ethanol, 20 volume % Cremophor EL, and 30 volume % propylene glycol. Dehydrated ethanol (USP, 500 mL, 394.5 g) was mixed with Cremophor EL (BASF Aktiengesellschaft, 200 mL, 210 g). After the foregoing two components were mixed to form a homogeneous liquid, propylene glycol (USP, 300 mL, 310.8 g) was added. The combination was mixed again to homogeneity and filtered through a 0.22μ filter, to provide 1 liter of vehicle.

### EXAMPLE 2

Following the general procedure of Example 1, another 1 L-batch of vehicle was prepared, using 450 mL (355.1 g) of ethanol, 280 mL (294 g) of Cremophor EL, and 270 mL (279.5 g) of propylene glycol. This resulted in a vehicle consisting of 45 volume % ethanol, 28 volume % Cremophor EL, and 27 volume % propylene glycol.

### EXAMPLE 3

Following the general procedure of Example 1, additional 1 L-batches of vehicle were prepared, using 500 mL (394.5 g) of ethanol and 150 to 500 mL (157.5 to 525 g) of Cremophor EL, and 0 to 350 mL propylene glycol. This resulted in vehicles consisting of 50 volume % ethanol, 15 to 50 volume % Cremophor EL, and 0 to 35 volume % propylene glycol.

### EXAMPLE 4

This example describes the preparation of a pharmaceutical solution formulation of this invention using a vehicle prepared in the preceding examples. 17-AAG (1.0 g) was accurately weighed out with an analytical balance into a clean glass container. Vehicle (95 mL) was added to the container and stirred until the 17-AAG was completely dissolved. The final volume of the solution was adjusted to 100.0 mL with additional vehicle. The solution was then filtered through a 0.22μ filter to ensure sterility and stored at 4° C.

### EXAMPLE 5

The stability of pharmaceutical solution formulations of this invention was demonstrated as follows. Two sets of sample formulations according to Example 1 were stored at 5° C. (“Sample A”) and 25° C. (“Sample B”), respectively. An aliquot of each sample was taken at Day 0, Day 17 and Day 23 and diluted to a final theoretical concentration of 400 μg/mL 17-AAG. The purity of and 17-AAG concentration in each aliquot were measured by reverse phase HPLC. The results are provided in Table A:

TABLE A
Stability of 17-AAG Formulation

17-AAG*

| Sample | Day | Concentration (μg/mL) | Purity (%) |
|---|---|---|---|
| A (5° C.) | 0 | 10.53 | 97.62 |
| | 17 | 10.97 | 96.93 |
| | 23 | 10.39 | 96.89 |
| B (25° C.) | 0 | 10.53 | 97.62 |
| | 17 | 10.88 | 96.28 |
| | 23 | 10.19 | 96.10 |

*Data is average of four samples

Longer term stability data for formulations of this invention are provided in Table B.

| Storage Conditions | Time (months) | Purity (%) |
|---|---|---|
| −20 ± 5° C. | 0 | 98.4 |
| | 1 | 98.9 |
| | 2 | 98.9 |
| | 3 | 98.7 |
| | 6 | 98.5 |
| | 9 | 98.1 |
| | 12 | 98.2 |
| 5 ± 3° C. | 0 | 98.4 |
| | 1 | 98.9 |
| | 2 | 98.8 |
| | 3 | 98.7 |
| | 6 | 97.4 |
| | 9 | 96.7 |
| | 12 | 95.9 |
| 25 ± 3° C. | 0 | 98.4 |
| 60 ± 5% Relative | 1 | 96.9 |
| Humidity | 2 | 95.7 |
| | 3 | 93.9 |
| | 6 | 87.1 |
| | 9 | 77.5 |
| | 12 | 71.9 |

The above results show that formulations of this invention are stable, even when stored at ambient temperature (though storage under refrigeration is recommended), for a period of at least three weeks or longer.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

All the documents cited in this specification are incorporated herein by reference.

1. A pharmaceutical solution formulation comprising 17-AAG in an amount of up to 15 mg/mL dissolved in a vehicle comprising (i) a first component that is ethanol, in an amount of between about 40 and about 60 volume %; (ii) a second component that is a polyethoxylated castor oil, in an amount of between about 15 to about 50 volume %; and (iii) a third component that is selected from the group consisting of propylene glycol, PEG 300, PEG 400, glycerol, and combinations thereof, in an amount of between about 0 and about 35 volume %.

2. A pharmaceutical solution formulation according to claim 1, wherein the second component is Cremophor EL.

3. A pharmaceutical solution formulation according to claim 1, wherein the third component is propylene glycol.

4. A pharmaceutical solution formulation according to claim 1, wherein the vehicle comprises the first component in an amount of about 45 to about 50 volume %, the second component in an amount of between about 20 to about 30 volume %, and the third component in an amount of between about 20 and about 30 volume %.

5. A pharmaceutical solution formulation according to claim 4, wherein the second component is Cremophor EL and the third component is propylene glycol.

6. A pharmaceutical solution formulation according to claim 1, wherein the vehicle comprises about 50 volume % ethanol, about 20 volume % Cremophor EL, and about 30 volume % propylene glycol.

7. A pharmaceutical solution formulation according to claim 4, wherein the vehicle comprises about 45 volume % ethanol, about 28 volume % Cremophor EL, and about 27 volume % propylene glycol.

8. A pharmaceutical solution formulation according to claim 1, wherein the third component is absent.

9. A method for administering 17-AAG to a patient in need thereof, comprising the steps of:
(a) providing a pharmaceutical solution formulation comprising 17-AAG in concentration of up to 15 mg/mL dissolved in a vehicle comprising (i) a first component that is ethanol, in an amount of between about 40 and about 60 volume %; (ii) a second component that is a polyethoxylated castor oil, in an amount of between about 15 to about 50 volume %; and (iii) a third component that is selected from the group consisting of propylene glycol, PEG 300, PEG 400, glycerol, and combinations thereof, in an amount of between about 0 and about 35 volume %;
(b) diluting the pharmaceutical solution formulation of step (a) into water to provide a diluted solution containing up to 3 mg/mL 17-AAG; and
(c) administering the diluted solution of step (b) intravenously to a patient.

10. A method according to claim 9, wherein the second component is Cremophor EL.

11. A method according to claim 9, wherein the third component is propylene glycol.

12. A method according to claim 9, wherein the vehicle comprises the first component in an amount of about 45 to about 50 volume %, the second component in an amount of between about 20 to about 30 volume %, and the third component in an amount of between about 20 and about 30 volume %.

13. A method according to claim 12, wherein the second component is Cremophor EL and the third component is Cremophor EL.

14. A method according to claim 9, wherein the vehicle comprises about 50 volume % ethanol, about 20 volume % Cremophor EL, and about 30 volume % propylene glycol.

15. A method according to claim 9, the vehicle comprises about 45 volume % ethanol, about 28 volume % Cremophor EL, and about 27 volume % propylene glycol.

16. A method according to claim 9, wherein the third component is absent.

17. A method according to claim 9, wherein the 17-AAG is administered in an amount from about 4 mg/m² to about 4000 mg/m².

18. A method according to claim 9, wherein the 17-AAG is administered in an amount of about 450 mg/m² weekly.

19. A method according to claim 9, wherein the 17-AAG is administered in an amount of about 308 mg/m² weekly.

20. A method for preparing a pharmaceutical solution formulation comprising 17-AAG, comprising the steps of:
(a) providing an amount of 17-AAG in a container;
(b) combining the 17-AAG of step (a) with an amount of a vehicle comprising (i) a first component that is ethanol, in an amount of between about 40 and about 60 volume %; (ii) a second component that is a polyethoxylated castor oil as a, in an amount of between about 15 to about 50 volume %; and (iii) a third component that is selected from the group consisting of propylene glycol, PEG 300, PEG 400, glycerol, and combinations thereof, in an amount of between about 0 and about 35 volume %;
(c) stirring the combination from step (c) until the 17-AAG is substantially dissolved; and
(d) optionally filtering the stirred combination from step (c) to form a pharmaceutical solution formulation comprising 17-AAG;
the amount of 17-AAG in step (a) and the amount of vehicle in step (b) being such that the concentration of 17-AAG in the pharmaceutical solution formulation is up to 15 mg/mL.

21. A method according to claim 20, wherein the second component is Cremophor EL.

22. A method according to claim 20, wherein the third component is propylene glycol.

23. A method according to claim 20, wherein the vehicle comprises the first component in an amount of about 45 to about 50 volume %, the second component in an amount of between about 20 to about 30 volume %, and the third component in an amount of between about 20 and about 30 volume %.

24. A method according to claim 23, wherein the second component is Cremophor EL and the third component is Cremophor EL.

25. about 20 volume % Cremophor EL, and about 30 volume % propylene glycol.

26. A method according to claim 20, the vehicle comprises about 45 volume % ethanol, about 28 volume % Cremophor EL, and about 27 volume % propylene glycol.

27. A method according to claim 20, wherein the third component is absent.
